(19) United States
(12) Patent Application Publication
(10) Pub. No.: US 2006/0030878 A1
(43) Pub. Date: Feb. 9, 2006

(54) Radiopaque distal embolic protection device

(75) Inventors: Kent D. Anderson, Champlin, MN (US); Jeannine B. Baden, Long Lake, MN (US); Kelly J. Huettl, Circle Pines, MN (US); Richard S. Kusleika, Eden Prairie, MN (US)

Correspondence Address: POPOVICH, WILES & O'CONNELL, PA;650 THIRD AVENUE SOUTH, SUITE 600, MINNEAPOLIS, MN, 55402, US

(73) Assignee: ev3 Inc.

(21) Appl. No.: 11/242,398

(22) Filed: Oct. 3, 2005

Related U.S. Application Data
(63) Continuation of application No. 10/165,803, filed on Jun. 7, 2002.
(60) Provisional application No. 60/296,647, filed on Jun. 7, 2001.

(51) Int. Cl. A61M 29/00

(52) U.S. Cl. 606/200

(57) ABSTRACT

The present invention is a radiopaque distal embolic protection device for use in a lumen of a patient's body, such as a blood vessel. The protection device has an expandable and retractable filter attached to a distal portion of a guidewire. At least a portion of the filter has a radiopaque coating for viewing under fluoroscopy during use. The radiopaque coating allows the operator to ensure that the periphery of the filter has fully engaged the wall of a blood vessel and to take appropriate measures in recovery of the protection device after capture of emboli and particulate matter.

### BACKGROUND OF THE INVENTION

1. Field of the Invention

The present invention relates generally to the field of embolic protection devices and vascular filters. More specifically, the present invention relates to a radiopaque embolic protection device.

2. Description of Related Art

Protection devices, such as embolic protection devices, are increasingly used in vascular intervention procedures. A protection device is an expandable and collapsible filter used to prevent the passage of particulate material, such as emboli, during a minimally invasive medical procedure. The protection device filter is moveably attached to a guidewire. In the collapsed configuration, the protection device can be advanced through a lumen of a patient's body, such as a blood vessel, to the treatment site. Once at the treatment site, the protection device is expanded such that the periphery of the protection device engages the wall of the lumen. Angioplasty, atherectomy, thrombectomy, laser ablation and/or stenting procedures may then be performed on the treatment site, and any particulate matter generated is prevented from entering the lumen of the patient's body distal to the position of the protection device. The protection device acts to prevent particulate matter from traveling to other parts of the patient's vascular system and causing a blockage or otherwise adversely affecting the peripheral areas of the vascular system.

Difficulties can arise where the protection device is not properly placed within the lumen. For example, if the periphery of the protection device does not fully engage the lumen wall, leaving a gap, then particulate matter might pass beyond the protection device. Also, when the protection device is being advanced or withdrawn from the lumen it may engage with an obstruction. The obstruction may be a stent that has been placed in a blood vessel, or an area of plaque build-up. The operator of the protection device may have to use different techniques depending upon the cause of the engagement. Thus, it would be advantageous to the operator to be able to find the exact location of the protection device within the lumen.

After the medical or diagnostic procedure is performed, the embolic protection device is recovered into a catheter. One problem that can occur upon recovery is that the protection device may become engaged or otherwise obstructed by a stent or other jagged or ensnaring region that may be present within a blood vessel, such as a stenosis or an area of plaque build-up. Another problem that can occur on recovery is that the protection device may not fully return to the retracted state due to a large amount of emboli and/or particulate matter captured within the protection device. The methods for recovering the protection device differ depending on the cause of the difficulty. For example, if the protection device is engaged with a stent, the operator may advance the protection device distally and then withdraw the protection device proximally so as to pass the stented region without becoming ensnared. If the protection device is not fully retracted into the recovery catheter due to a large amount of emboli captured, the operator may decide to substitute a larger recovery catheter or to aspirate some debris and then draw the protection device into the catheter, or to recover the protection device when it is only partially enclosed in the catheter.

The current art employs radiopaque materials or coatings applied to guidewires and stents. Radiopaque materials allow the operator to view the position of the marked material using fluoroscopy. This has been used for proper positioning of a guidewire within a lumen, and in positioning of stents. As it applies to protection devices, a radiopaque marker band has been located on a guidewire adjacent to a protection device. A marker band is a radiopaque band that surrounds the circumference of a guidewire or catheter so that the location can be determined on the fluoroscopy.

Medical devices that incorporate a radiopaque coating can be viewed under fluoroscopy by an operator, such as a doctor, during operation of the device within the blood vessel.

U.S. Pat. No. 6,203,561 B1, Ramee, discloses a protection device with a support hoop having a radiopaque band, wherein the support hoop forms the mouth of a blood permeable sac. There are some shortcomings to the Ramee device. One is that Ramee teaches the use of radiopaque bands only about the support hoop of the sac.

The prior art also discloses protection devices which include a plurality of filaments expandable outwardly from a guidewire. The filaments are moveable with respect to each other such that they may conformingly engage a non-uniform lumen wall. However, such disclosed devices have radiopaque marker bands mounted to a guidewire proximate the device and/or to struts of a frame of the device. See EP 1,172,073 FIG. 32A. These devices do not employ a radiopaque filter structure, however, wherein mesh of a device basket is itself radiopaque. This prevents an operator from viewing the periphery under fluoroscopy to ensure that the periphery has fully engaged the lumen wall. Also, there are filter frames that expand a mesh or perforated film. A radiopaque strand is placed within the mesh or wrapped around a portion of the mesh to provide radiopacity to the mesh. See Gilson, U.S. Pat. No. 6,336,934 FIG. 36 and U.S. Pat. No. 6,066,149. This construction requires additional components to be added to the filter body. The mesh or perforated film that form the body of the filter are not radiopaque.

### SUMMARY OF THE INVENTION

The present invention is an embolic protection device having a radiopaque device mesh structure that is expandable about a distal portion of a guidewire. The device mesh has a plurality of filaments mounted with respect to the guidewire such that the filaments expand radially outwardly from the guidewire. The filaments cross and intersect one another so as to form the filter protection device. The expanded filter device has a lip or mouth-defining portion that forms an entry periphery through which emboli enter the filter body of the device. The periphery of the device is the most radial outward portion of the lip which engages a wall of a lumen in a patient.

The present invention is intended for use in a lumen of a patient's body such as a blood vessel. Radiopaque filaments allow the filter to be viewed under fluoroscopy during a medical procedure. To achieve this, the filter is first advanced within the vascular system using the guidewire. The filter is maintained in a retracted configuration until properly positioned for deployment. Once the filter is deployed, the operator can ensure, in view of the radiopacity of the filaments, that the filter has properly engaged the lumen wall of the blood vessel and that the filter is properly sized for the blood vessel. Because the filaments are flexible and moveable with respect to each other, the filter is flexible and is deployable in diseased areas or within other non-uniform sections of a lumen such as a bend. Using fluoroscopy, an operator can ensure that the periphery has properly engaged an irregularly shaped lumen wall.

The present invention is configured and constructed so as to provide radiopacity to a deployable and retractable filter for ensuring the filter has engaged a lumen wall and assisting in recovery of the filter after the performance of a medical procedure.

One embodiment of the present invention is a radiopaque filter wherein the filaments forming the filter are radiopaque.

Another embodiment of the present invention is a filter wherein a portion of the filter filaments are radiopaque.

Another embodiment of the present invention is a filter wherein a preselected number of the filter filaments are radiopaque.

Another embodiment of the present invention is a filter wherein the periphery of the filter is radiopaque.

Another embodiment of the present invention is a filter wherein a radiopaque coating is applied to at least a portion of a selected number of filaments.

Another embodiment of the present invention is a filter wherein at least a portion of the filaments have a radiopaque clad composite structure.

Another embodiment of the present invention is a filter wherein an adhesion or tie layer is disposed between a filament surface and a radiopaque coating.

Still another embodiment of the present invention is a device wherein a drug is applied and/or incorporated with the radiopaque filter filaments for providing anti-thrombogenic properties to the filter.

The present invention also includes a method of making a filter device having radiopaque filaments.

The present invention also includes a method of viewing the filter device under fluoroscopy for ensuring filter contact with a lumen wall during a medical procedure.

The present invention also includes a method of enabling recovery of the filter after the medical procedure.

### BRIEF DESCRIPTION OF THE DRAWINGS

FIG. 1 is a side elevational view of a partially deployed protection device with radiopaque filter filaments;

FIG. 2 is a perspective view of a fully expanded filter having a radiopaque coating;

FIG. 3 is an enlarged detail view from the area encircled at 3 in FIG. 1 of radiopaque filaments;

FIG. 4 is a view, similar to FIG. 3, illustrating in an exaggerated fashion, a filter with a portion of each of the filaments being radiopaque;

FIG. 5 is a view, similar to FIG. 3 illustrating in an exaggerated fashion an expanded filter with the full lengths of the filaments being radiopaque;

FIG. 6 is a view similar to FIG. 3 illustrating in an exaggerated fashion an expanded filter with selected filaments being radiopaque;

FIG. 7 is an enlarged fragmentary perspective view of a filament having a radiopaque coating;

FIG. 8 is an enlarged fragmentary perspective view of a filament having an adhesion layer between the filament surface and a radiopaque coating; and

FIG. 9 is an enlarged fragmentary perspective view of a filament having a drug coating applied over a radiopaque coating.

### DESCRIPTION OF THE PREFERRED EMBODIMENTS

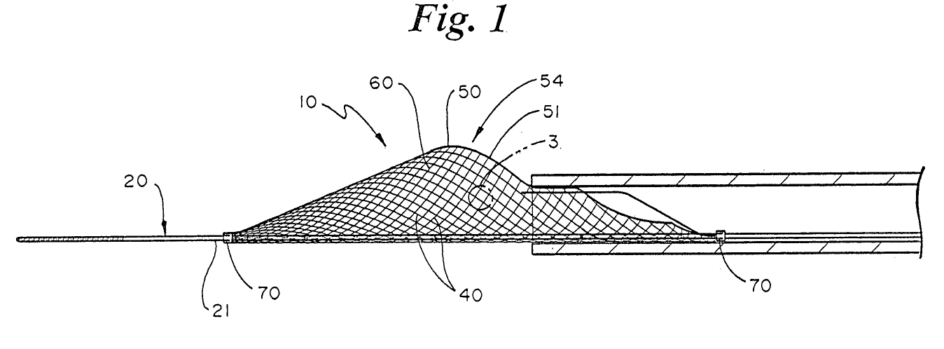

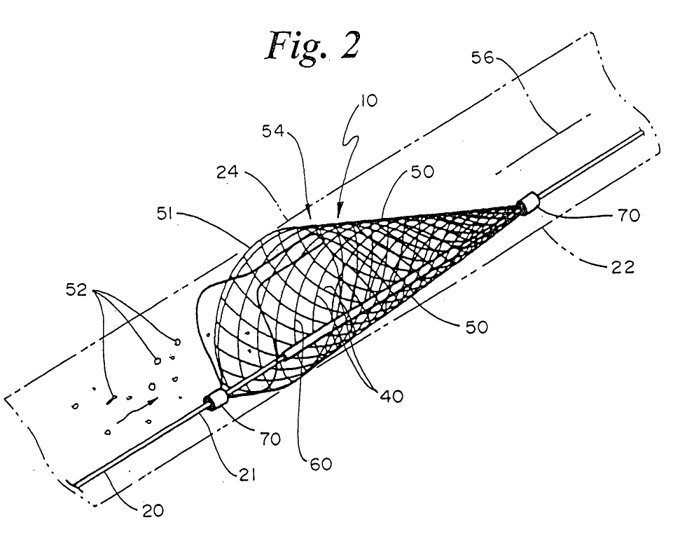

FIGS. 1 and 2 illustrate a protection device or filter 10 mounted to a guidewire 20. The guidewire 20 is an elongate member having a distal portion 21. The filter 10 is mounted at the distal portion 21 of the guidewire 20. The filter 10, shown partially deployed, may assume an expanded or a retracted configuration depending upon whether it is constrained by a catheter. In the expanded configuration, the filter 10 extends radially outward about an axis 56 to form a periphery. The periphery, illustrated at 50, is defined by the outermost portion of the filter 10. In the embodiment shown, a lip 51 is at least partially axially coincident with periphery 50 and defines a mouth to allow the capture of emboli 52 within the filter 10. The filter 10 may optionally have one or more radiopaque marker bands 70.

Suitable filters with respect to which concepts according to the present invention can be employed include those disclosed in WO 96/01591, U.S. Pat. No. 6,325,815, WO 01/15629 and EP 1,181,900, the disclosures of which are hereby incorporated by reference.

The filter 10 may have a basket shape 54 as illustrated or one of a variety of other shapes that allow for the filter function to be performed. The embodiment in FIG. 1 includes a proximal lip 51 formed by ends of a plurality of filaments 40. The filaments 40 are expandable and collapsible about an axis of elongation. The lip 51 facilitates receipt of emboli and particulate matter 52 within the filter 10.

At least a portion of the filter 10 is radiopaque, referred to as 60. Several embodiments of a radiopaque filter are contemplated. For example, the entire length of a filament 40 may be radiopaque, only a portion of a filament 40 may be radiopaque, an intermittent pattern of radiopaque and non-radiopaque filaments may be employed, selected filaments may be radiopaque in full or in part. Any combination resulting in at least a portion of the filter 10 being radiopaque is contemplated according to the present invention. The radiopaque portion of the filter is illustrated as the shaded area in the various figures as 60. The radiopaque portion 60 allows the viewing of the filter 10 under fluoroscopy to ascertain the spatial relationship between the filaments 40 of the filter 10, the guidewire 20, and the patient's body or other interventional devices and implants.

In a retracted or collapsed configuration constrained by a catheter, the periphery of the filter 10 is disposed radially inward toward the guidewire 20. With the filter 10 in a retracted configuration, the guidewire 20 can be advanced within a lumen such as a blood vessel of a patient's body. In the expanded configuration, the periphery is intended to engage the wall of the lumen so as to filter a fluid such as blood flowing within the lumen.

FIG. 2 illustrates the filter 10 in the expanded configuration. The filter 10 comprises a plurality of filaments 40. The filaments 40 typically intersect and cross other filaments 40 so as to define a multiplicity of pores within the filter 10. The filaments 40 are flexible and moveable or slidable with respect to one another and with respect to the guidewire 20. In the expanded state, the filaments 40 define a periphery of the filter 10 which will conformingly engage the lumen wall.

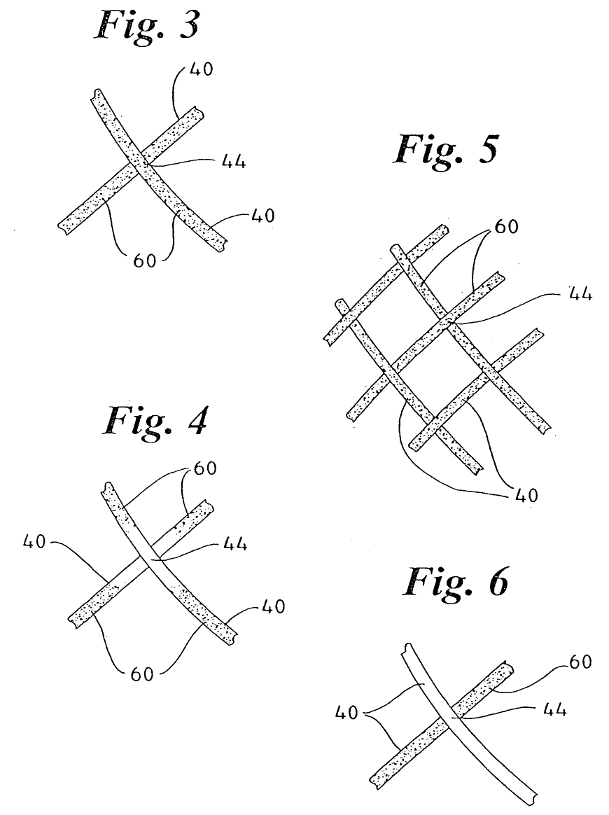

Embodiments of the present invention include the various radiopaque filaments 40 as illustrated in FIGS. 3-6. Medical devices that are radiopaque 60 can be viewed under fluoroscopy by an operator, such as a doctor, during operation of the device within the blood vessel. The filament may be radiopaque 60 over the entire length of a filament 40, as in FIG. 5, or over a selected portion of the filament 40, as in FIG. 4. The filaments 40 may be radiopaque at the intersection 44 with another filter as in FIG. 3, or may be radiopaque only on a portion adjacent the intersection, as in FIG. 4. Alternatively or in combination, only a preselected number of filaments 40 are radiopaque, as in FIG. 6. These embodiments or any combination are hereby incorporated by the present invention.

The radiopaque filaments may be made by coating the filaments with a radiopaque coating or by using filaments comprising a clad composite material that is radiopaque.

In making a device of the present invention using a radiopaque coating, the coating 60 may be applied to the filter 10 while in the expanded or retracted configuration. The filter 10 may be cycled by alternating between the retracted and expanded state. It is preferable that the filter 10 be cycled during coating so as to maintain flexibility at the areas where filaments 40 cross each other. Such cycling may be performed during coating or after coating and/or prior to performing a medical procedure. This cycling may prevent the radiopaque coating 60 from immobilizing a wire or filament intersection 44. The coating 60 should be applied so as not to disable the filter 10 from freely expanding outwardly and collapsing to the retracted state.

The radiopaque coating 60 allows the filter 10 to be viewed under fluoroscopy during use in a vascular system. The guidewire 20 may be used to advance the filter 10 within the vascular system. The radiopaque coating 60 helps the viewer to ensure proper positioning of the filter 10 within the lumen before deploying the filter 10 from the collapsed state to the expanded state. The filter 10 is then expanded within the lumen. The radiopaque portion 60 illustrating the periphery 50 can be determined to ensure that the entire lumen wall has been engaged by the lip 51 defining the mouth. If the fluoroscopy indicates that the filter 10 has not properly engaged the lumen wall, the filter 10 may be withdrawn into the deployment catheter and re-deployed. Alternatively, the viewing of the filter 10 may indicate that a different filter size would be appropriate, and, in such a case, the filter 10 can be removed from the lumen and replaced with an appropriately sized filter 10.

Alternatively, the radiopaque filaments 40 may comprise a radiopaque core of clad composite structures such as tantalum, platinum, or gold. One source of such material is Ft. Wayne Metals, and is known as Drawn Filled Tubing (DFT). The filter 10 may be entirely comprised of filaments having clad composite structures. Alternatively, the filter 10 may have a selected or predetermined number of filaments having a radiopaque core. A radiopaque coating may be used on filters having filaments with clad composite structures.

FIGS. 1-2 also illustrate a filter 10 of the present invention. The filter 10 is in an expanded configuration. The periphery 50 is defined by the multiplicity of filaments 40 expanded about an axis of elongation. The periphery 50 is, it is intended, able to conformingly engage a wall of a lumen. At least a portion of the filaments 40 illustrated in FIG. 1 would be radiopaque 60 to enable viewing the position and configuration of the filter 10 under fluoroscopy.

The periphery 50 of the filter 10 is defined by a proximally facing lip 51. The filaments 40 are flexible and moveable with respect to each other such as during expansion and retraction of the filter 10. The filter 10 may be expanded within a portion of a lumen or at a bend or turn in the vascular system. The flexibility of the filaments 40 allows the periphery 50 of the filter 10 to adapt and conform to such an irregularly shaped lumen wall. The radiopacity of the filter 10 ensures that the periphery 50 properly engages the lumen wall, regardless of the shape of the wall.

FIG. 3 illustrates the intersection 44 of two filaments 40 of the filter 10. The intersecting portion of the filaments 40 are radiopaque 60.

FIG. 5 illustrates a filter 10 having filaments, at least a portion of which are radiopaque. The radiopaque portion 60 is illustrated by the shaded area. The radiopaque portion 60 is shown as including filament intersections 44.

FIG. 4 illustrates a radiopaque portion applied only to portions of the filaments 40 that do not comprise the intersections 44 of filaments 40. The radiopaque coating portion is illustrated as the shaded area.

FIG. 6 illustrates a radiopaque filament 60 intersecting with a non-radiopaque filament 40. It is contemplated by the present invention that a portion of the filaments of the filter 10 may be radiopaque 60 whereas the remaining filaments need not be radiopaque. Alternatively, the remaining filaments may have a portion that is radiopaque.

It will be understood that the entire filter or only a portion of the filter may be radiopaque according to the present invention. For example, the periphery or only a portion of the periphery may be radiopaque to accomplish the purposes of the present invention. For example, the periphery 50 of the filter 10 may have intervals that are radiopaque and adjacent intervals that are not.

Once the filter 10 is expanded to properly engage the lumen, the diagnostic procedure and/or medical treatment may be performed. These may include stenting, ablation, angioplasty and the like. The filter 10 will prevent the passage of particulate matter from flowing distal to the filter 10 during the procedure by capturing loose emboli within the filter 10.

After the site has been treated, the filter 10 can be retracted and recovered from the blood vessel. A number of problems may occur during filter 10 recovery. The filter 10 may have trapped a large amount of emboli. The emboli may prevent the filter 10 from being able to collapse so as to allow recovery of the filter 10 within a recovery catheter. Another problem is that the filter 10 may become ensnared on a stent or other such obstruction within the lumen so as to prevent the filter 10 from further advancement within the lumen. An operator will be able to distinguish these situations and other problems by viewing the filter 10 under fluoroscopy. In the case of the former problem, for example, the operator will be able to visually observe that the filter 10 has not been fully retracted. In the case of the latter problem, the operator will be able to visually ascertain whether the filter 10 is engaged with an obstruction or is not fully retracted. The radiopacity allows the operator to distinguish between these and other situations that might prevent the recovery of the filter 10. Once the impediment has been identified, the operator can take appropriate measures to recover the filter 10. Such measures may differ depending upon the cause of the ensnarement. It is the radiopacity of the filter 10 that allows an operator to view the operation of the filter 10 for appropriately assessing a course of action.

If the filter is entangled, engaged or obstructed, the operator may view and assess the obstruction under fluoroscopy and advance the filter so as to avoid the obstruction. The filter may instead be unable to be fully retracted due to the amount of emboli captured therewithin. The operator can view this condition under fluoroscopy and aspirate the lumen with a catheter so as to remove a portion of the emboli from the filter. A different sized catheter may be required to properly aspirate the lumen. Alternatively, the operator may decide to recover the filter containing debris by not fully drawing the filter with debris into the recovery catheter but rather by allowing a distal portion of the filter with debris therein to remain outside of and distal to the catheter while the catheter/filter/debris are withdrawn as a unit.

The radiopaque coating 60 may be a metal, polymer, ceramic, radiolucent mesh or composite coating or a combination of such materials. These coatings may be applied to the periphery, a portion thereof, the entire filter 10, a portion thereof, a plurality of filaments 40, a portion of a filament 40, a portion of the filaments, or any other such combination wherein at least a desired portion of the filter 10 is radiopaque 60.

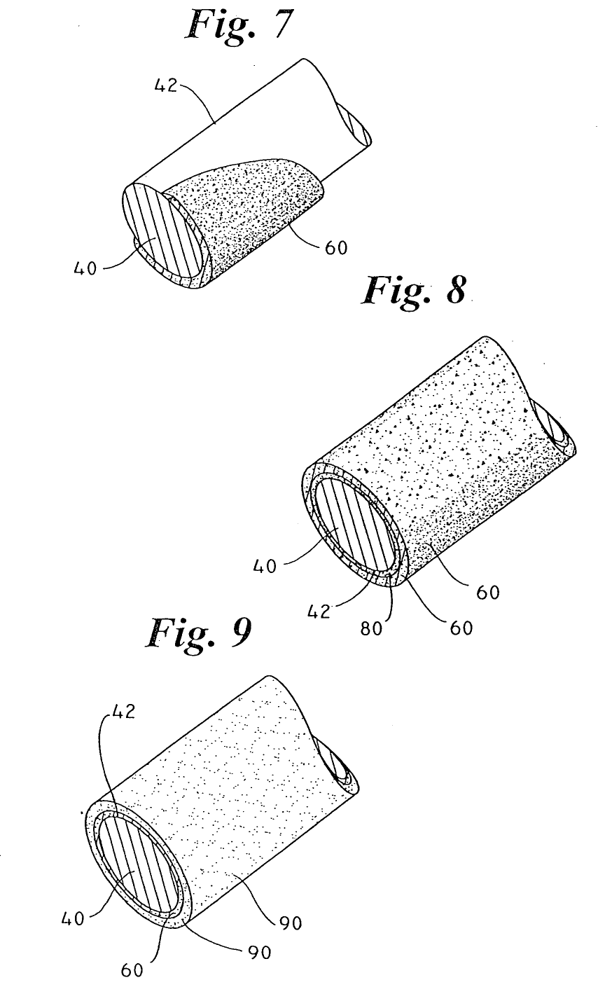

FIG. 7 illustrates a filament 40 having a radiopaque coating 60 thereon. The filament 40 is shown in the center and the radiopaque coating 60 surrounding at least a portion of the surface of the filament 40.

The filaments 40 may be a wire or shape memory alloy such as Nickel-Titanium. The filaments 40 may be afforded a predetermined configuration such as a helical or curved shape such that they are able to slidably intersect portions of other filaments 40. The filaments 40 should have a diameter of about 0.001 inches to about 0.010 inches, and more preferably from about 0.002 inches to about 0.0025 inches. Each filament 40 has a surface 42 along which the radiopaque coating 60 can be applied.

FIG. 8 illustrates a portion of a filament 40 having a radiopaque coating 60 wherein an adhesion layer 80 is interposed between the surface of the filament 42 and the coating 60. An adhesion layer 80 may be applied between the filament surface 42 and radiopaque coating 60 to securely maintain the coating 60 to the filament 40. The adhesion layer 80 acts as an adhesive between the radiopaque coating 60 and the filament 40. The adhesion layer 80 may cover all or a portion of the filament surface 42. The coating 60 may cover all or a portion of the adhesion layer 80 and all or a portion of the filament surface 42. In a preferred embodiment, the adhesion layer 80 has a thickness from about 90 Angstroms to about 3100 Angstroms. An example of an appropriate adhesion layer is a layer of titanium deposited on a sputter cleaned nitinol surface for adhering gold to nitinol. In one embodiment, the coating layer 60 has a thickness from about 3 microns to about 15 microns.

Examples of metals that can be used in radiopaque coatings include: gold, tin, platinum, tantalum, silver, titanium, nickel, zirconium, rhenium, bismuth, vanadium, chromium, iron, cobalt, copper, bromine, niobium, molybdenum, tungsten and the like, and combination alloys thereof. Combinations of non-metals or any other combination sufficient for providing radiopacity for effecting the purpose of the present invention are also appropriate. Visibility under fluoroscopy is greater with elements having atomic numbers greater than those of the elements found in the patient's body.

Polymeric compounds may be used to provide radiopaque coatings. Polymeric compounds may included a polymer matrix combined with a radiopaque agent. Such agents may include barium sulfate, an iodine containing agent such as OmniPaque.RTM, or any other agent suspended or added to the polymeric matrix in any appropriate way. The polymeric matrix may also include fillers such as tungsten powder, bismuth subcarbonate, bismuth oxycholoride, and any other filler known in the art.

The filter 10 as disclosed herein is generally used only temporarily within a patient's vascular system. A coating 60 having a temporary or limited radiopacity time may, therefore, be used as a result of the short term duration of the use of the filter 10. For example, a radiopaque coating 60 that maintains radiopacity for several hours may be sufficient for the functioning of the present invention.

Another embodiment of the present invention uses a radiopaque polymer film applied to a surface or adhesion layer on a filament 40 or portion thereof. The polymer film may contain gold particles such as spherical gold particles or gold particles mixed with a heparin solution for increased anti-coagulation properties. The gold particle mixture may be suspended in a monomer polymer mixture. The polymer film may have embedded micro spheres acting as micro filters 10 for filtering of microparticles. The filter patency may be enhanced by filtering micro particles in the blood stream that are precursors to thrombosis and lead to filter occlusion.

In addition to providing radiopacity of the filter 10, the radiopaque coating 60 may contain or otherwise include a drug or drug coating for preventing coagulation or prolonging filter 10 patency. FIG. 9 illustrates a portion of a filament 40 having a radiopaque coating 60 and a drug coating 90 thereon. An example of such a drug coating might include covalently bonded heparin, micro encapsulated ticlopidine, a clot dissolving enzyme or an antiplatelet agent.

In addition to adding a radiopaque coating 60 to the filter 10, a radiopaque coating may be added to the guidewire 20.

It will be understood that this disclosure, in many respects, is only illustrative. Changes may be made in details, particularly in matters of shape, size, material, and arrangement of parts without exceeding the scope of the invention. Accordingly, the scope of the invention is as defined in the language of the appended claims.

1-29. (canceled)

30. A method of using a radiopaque protection device comprising the steps of:
a) advancing a protection device having a radiopaque filter to a predetermined position within a lumen;
b) expanding said radiopaque filter within said lumen; and
c) viewing said radiopaque filter under fluoroscopy to ensure said radiopaque filter engages said lumen.

31-36. (canceled)

37. A method of performing a vascular procedure in a patent comprising:
(a) providing an embolic protection device comprising an elongate member having a distal portion and a filter being expandable and collapsible about the distal portion, the filter comprising a multiplicity of filaments that form a mesh, the filaments comprising a metal, at least a portion of the mesh being radiopaque, and the filter having an open mouth;
(b) advancing the embolic protection device in a collapsed configuration through a lumen of the patient's body to a treatment site;
(c) expanding the filter;
(d) performing a vascular procedure at the treatment site, wherein particulate matter is generated; and
(e) collecting the particulate matter in the filter.

38. The method of claim 37, further comprising viewing the filter under fluoroscopy.

39. The method of claim 37, wherein the vascular procedure is selected from angioplasty, atherectomy, thrombectomy, laser ablation, or stenting.

40. The method of claim 37, wherein a majority of the filaments are radiopaque.

41. The method of claim 37, wherein the filaments have a radiopaque coating.

42. The method of claim 37, wherein the filaments have a radiopaque core.

43. The method of claim 41, wherein the radiopaque coating overlies an adhesion layer.

44. The method of claim 41, wherein the radiopaque coating has an overlying drug coating.

45. The method of claim 44, wherein the drug coating has anti-coagulation properties.

46. The method of claim 41, wherein the radiopaque coating is a polymeric compound.

47. The method of claim 41, wherein the radiopaque coating is a polymer matrix.

48. The method of claim 41, wherein the radiopaque coating comprises a metal.

49. The method of claim 41, wherein the radiopaque coating comprises a ceramic.

50. The method of claim 41, wherein the radiopaque coating has temporary radiopacity.

51. The method of claim 41, wherein the radiopaque coating is a polymer film.

52. The method of claim 51, wherein the polymer film comprises embedded microspheres.

53. The method of claim 44, wherein the drug coating comprises covalently bonded heparin.

54. The method of claim 44, wherein the drug coating comprises an antiplatelet agent.

55. The method of claim 37, wherein the filaments are made entirely of metal.

56. The method of claim 55, wherein the filaments comprise a nickel-titanium shape memory alloy.

57. The method of claim 37, wherein a periphery of the filter conformingly engages a wall of the lumen when the filter is expanded.

58. A method of claim 38, wherein the filter is viewed under fluoroscopy after it has been expanded to ensure that the filter engages the lumen.

59. A method of claim 38, further comprising (i) retracting the device within the lumen; (ii) viewing the device under fluoroscopy to assess an obstruction; and (c) handling the device to overcome the obstruction.

60. The method of claim 38, further comprising aspirating the lumen with a catheter to remove at least a portion of the particulate matter.

61. The method of claim 38, further comprising determining a device recovery strategy based at least in part on an image of the protection device under fluoroscopy.
